(19) United States
(12) Patent Application Publication
(10) Pub. No.: US 2009/0131792 A1
(43) Pub. Date: May 21, 2009

(54) ULTRASONIC DIAGNOSIS APPARATUS

(75) Inventor: Tsutomu Yawata, Tokyo (JP)

Correspondence Address: PATRICK W. RASCHE (20459);ARMSTRONG TEASDALE LLP, ONE METROPOLITAN SQUARE, SUITE 2600, ST. LOUIS, MO, 63102-2740, US

(73) Assignee: GE MEDICAL SYSTEMS GLOBAL TECHNOLOGY COMPANY, LLC, Waukesha, WI (US)

(21) Appl. No.: 12/263,052

(22) Filed: Oct. 31, 2008

(30) Foreign Application Priority Data
Nov. 2, 2007 (JP) 2007-285618

(51) Int. Cl. A61B 8/00

(52) U.S. Cl. 600/441

(57) ABSTRACT

An ultrasonic diagnosis apparatus includes an image display device for displaying an image generated based on ultrasonic echo signals received from an subject on a screen, a trace pointer display device for displaying a trace pointer on the screen on which the image is being displayed during a trace measurement, and an operating device for allowing an operator to perform an action to instruct to start a trace measurement, an action to move the trance pointer on the screen, and an action to instruct to terminate a trace measurement. The image display device performs a zoomed-in display of an image portion in a section around the trace pointer.

### CROSS REFERENCE TO RELATED APPLICATIONS

This application claims the benefit of Japanese Patent Application No. 2007-285618 filed Nov. 2, 2007, which is hereby incorporated by reference in its entirety.

### BACKGROUND OF THE INVENTION

The subject matter disclosed herein relates to ultrasonic diagnosis apparatus and, more particularly, to an ultrasonic diagnosis apparatus that can improve the workability of manually tracing the contour of an image and the like displayed on screen.

Ultrasonic diagnosis apparatus that provides manually or automatically tracing Doppler spectral waveforms displayed on screen has heretofore been known (e.g., refer to Japanese Unexamined Patent Publication No. 2005-81081).

When manually tracing Doppler spectral waveforms, a work of moving a trace pointer along the Doppler spectral waveforms is performed by, for example, rolling a track ball.

In the above ultrasonic diagnosis apparatus of prior art, however, if Doppler spectral waveforms are displayed at a smaller scale factor to bring several cycles of waveforms into view, it is easy to find where the trace pointer is now positioned in the entire Doppler spectral waveforms, but it is difficult to see whether the trace pointer accurately traces a waveform at the moment. On the other hand, if the waveforms are displayed at a larger scale factor, it is easy to see whether the trace pointer accurately traces one of the Doppler spectral waveforms at the moment, but it is difficult to find where the trace pointer is positioned in the entire Doppler spectral waveforms. That is, poor workability of manually tracing the Doppler spectrum was a problem.

### BRIEF DESCRIPTION OF THE INVENTION

It is desirable that the problem described previously is solved.

In a first aspect, the invention provides an ultrasonic diagnosis apparatus including: an image display device for displaying an image generated based on ultrasonic echo signals received from an subject on a screen; a trace pointer display device for displaying a trace pointer on the screen on which the image is being displayed during a trace measurement; and an operating device for allowing an operator to perform an action to instruct to start a trace measurement, an action to move the trance pointer on the screen, and an action to instruct to terminate a trace measurement, characterized in that the image display device performs a zoomed-in display of an image portion in a section around the trace pointer.

In the ultrasonic diagnosis apparatus according to the first aspect, because an image portion in the section around the trace pointer is displayed at a higher scale factor than other parts of the image, it is easy to see whether the trace pointer accurately traces the image contour or the like at the moment. Because image portions before and after the section around the trace pointer are displayed at a lower scale factor than the scale factor applied in the section around the trace pointer, it is easy to find where the trace pointer is now positioned in the entire image. Consequently, it is possible to improve the workability of manually tracing the contour of an image and the like displayed on screen.

In a second aspect, the invention provides the ultrasonic diagnosis apparatus according to the first aspect, characterized in that the action to instruct to start a trace measurement is pressing a function key.

In the ultrasonic diagnosis apparatus according to the second aspect, the instruction to start a trace measurement can be given by using a function key.

In a third aspect, the invention provides the ultrasonic diagnosis apparatus according to the first or second aspect, characterized in that the action to move the trace pointer is rolling a track ball.

In the ultrasonic diagnosis apparatus according to the third aspect, the trace pointer can be moved by using the track ball.

In a fourth aspect, the invention provides the ultrasonic diagnosis apparatus according to any of the first through third aspects, characterized in that the action to terminate a trace measurement is pressing a function key.

In the ultrasonic diagnosis apparatus according to the fourth aspect, the instruction to terminate a trace measurement can be given by using a function key.

In a fifth aspect, the invention provides the ultrasonic diagnosis apparatus according to any of the first through fourth aspects, characterized in that the area of the zoomed-in display of an image portion moves, as the trace pointer moves.

In the ultrasonic diagnosis apparatus according to the fifth aspect, when the trace pointer is moved, the accompanying displacement of the area of the zoomed-in display of an image portion automatically takes place. Therefore, it is possible to improve the workability.

In a sixth aspect, the invention provides the ultrasonic diagnosis apparatus according to any of the first through fifth aspects, further including a zoomed-in display area changing device for allowing the operator to change the dimensions of the area of the zoomed-in display of an image portion.

In the ultrasonic diagnosis apparatus according to the sixth aspect, the area of the zoomed-in display of an image portion can be changed in accordance with difference in images and operator's preference.

In a seventh aspect, the invention provides the ultrasonic diagnosis apparatus according to any of the first through sixth aspects, characterized in that the area of the zoomed-in display of an image portion is a rectangular area including therewithin a position at which the trace pointer is displayed.

In the ultrasonic diagnosis apparatus according to the seventh aspect, because the area of the zoomed-in display of an image portion is a rectangle, the forward and rear boundaries of the area of the zoomed-in display of an image portion are apparent.

In an eighth aspect, the invention provides the ultrasonic diagnosis apparatus according to any of the first through seventh aspects, characterized in that an action to turn on/off a function for the zoomed-in display of an image portion can be performed by the operating device.

In the ultrasonic diagnosis apparatus according to the eighth aspect, performing the zoomed-in display of an image portion can be turned on/off arbitrarily and, therefore, it is possible to improve the workability.

In a ninth aspect, the invention provides the ultrasonic diagnosis apparatus according to the eighth aspect, characterized in that the action to turn on/off the function for the zoomed-in display of an image portion is pressing a function key.

In the ultrasonic diagnosis apparatus according to the ninth aspect, performing the zoomed-in display of an image portion can be turned on/off arbitrarily by using a function key.

In a tenth aspect, the invention provides the ultrasonic diagnosis apparatus according to any of the first through ninth aspects, characterized in that, besides the action to instruct to start a trace measurement and the action to instruct to terminate a trace measurement, an action to specify a trace start point and an action to specify a trace end point can be performed by the operating device.

In the ultrasonic diagnosis apparatus according to the tenth aspect, a trace start point can be specified with the trace pointer being accurately positioned on the image contour or the like. Also, a trace end point can be specified with the trace pointer being accurately positioned on the image contour or the like.

In an eleventh aspect, the invention provides the ultrasonic diagnosis apparatus according to the tenth aspect, characterized in that the action to specify a trace start point is pressing a button attached to the track ball.

In the ultrasonic diagnosis apparatus according to the eleventh aspect, the operator can specify a trace start point without releasing his or her hand from the track ball.

In an twelfth aspect, the invention provides the ultrasonic diagnosis apparatus according to the tenth or eleventh aspect, characterized in that the action to specify a trace end point is pressing a button attached to the track ball.

In the ultrasonic diagnosis apparatus according to the twelfth aspect, the operator can specify a trace end point without releasing his or her hand from the track ball.

In a thirteenth aspect, the invention provides the ultrasonic diagnosis apparatus according to any of the tenth through twelfth aspects, characterized in that the image display device starts to perform the zoomed-in display of an image portion, when the action to specify a trace start point is performed during an inactive state of the zoomed-in display of an image portion.

In the ultrasonic diagnosis apparatus according to the thirteenth aspect, the function for the zoomed-in display of an image portion can be turned on automatically by the action to specify a trace start point and it is possible to improve the workability.

In a fourteenth aspect, the invention provides the ultrasonic diagnosis apparatus according to any of the tenth through thirteenth aspects, characterized in that the image display device deactivates the zoomed-in display of an image portion, when the action to specify a trace end point is performed during an active state of the zoomed-in display of an image portion.

In the ultrasonic diagnosis apparatus according to the fourteenth aspect, the function for the zoomed-in display of an image portion can be turned off automatically by the action to specify a trace end point and it is possible to improve the workability.

In a fifteenth aspect, the invention provides the ultrasonic diagnosis apparatus according to any of the first through twelfth aspects, characterized in that the image display device starts to perform the zoomed-in display of an image portion, when the action to instruct to start a trace measurement is performed during an inactive state of the zoomed-in display of an image portion.

In the ultrasonic diagnosis apparatus according to the fifteenth aspect, the function for the zoomed-in display of an image portion can be turned on automatically by the action to instruct to start a trace measurement and it is possible to improve the workability.

In a sixteenth aspect, the invention provides the ultrasonic diagnosis apparatus according to any of the first through twelfth aspects or the fifteenth aspect, characterized in that the image display device deactivates the zoomed-in display of an image portion, when the action to instruct to terminate a trace measurement is performed during an active state of the zoomed-in display of an image portion.

In the ultrasonic diagnosis apparatus according to the sixteenth aspect, the function for the zoomed-in display of an image portion can be turned off automatically by the action to instruct to terminate a trace measurement and it is possible to improve the workability.

In a seventeenth aspect, the invention provides the ultrasonic diagnosis apparatus according to any of the first through sixteenth aspects, characterized in that the image is a Doppler spectrum.

In the ultrasonic diagnosis apparatus according to the seventeenth aspect, trace measurement on a Doppler spectrum can be performed.

In an eighteenth aspect, the invention provides the ultrasonic diagnosis apparatus according to any of the first through sixteenth aspects, characterized in that the image is a B-mode image.

In the ultrasonic diagnosis apparatus according to the eighteenth aspect, trace measurement for, for example, measuring the length around the head of a fetus can be performed.

According to the ultrasonic diagnosis apparatus of the invention, it is possible to improve the workability of manually tracing the contour of an image and the like displayed on screen.

Further objects and advantages of the present invention will be apparent from the following description of the preferred embodiments of the invention as illustrated in the accompanying drawings.

### BRIEF DESCRIPTION OF THE DRAWINGS

FIG. 1 is a perspective view depicting an ultrasonic diagnosis apparatus according to an embodiment.

FIG. 2 is a flowchart illustrating a trace measurement process according to the embodiment.

FIG. 3 depicts an example of a screen in which an image to be subjected to trace measurement is frozen.

FIG. 4 depicts an example of a screen in which the trace pointer is displayed.

FIG. 5 depicts an example of a screen in which the trace pointer has been moved to a trace start point.

FIG. 6 depicts an example of a screen in which an image portion in a section around the trace pointer is zoomed in and displayed.

FIG. 7 depicts an example of a screen in which a track of the trace pointer is shown in a bold line.

FIG. 8 depicts an example of a screen in which the result of trace measurement is displayed.

FIG. 9 depicts an example of a screen in which an image portion in a section around the trace pointer is zoomed in and displayed after zoom-in parameters are changed.

### DETAILED DESCRIPTION OF THE INVENTION

In the following, various embodiments of the invention will be described in greater detail by way of reference to the drawings. The embodiments described herein are not intended to limit the invention.

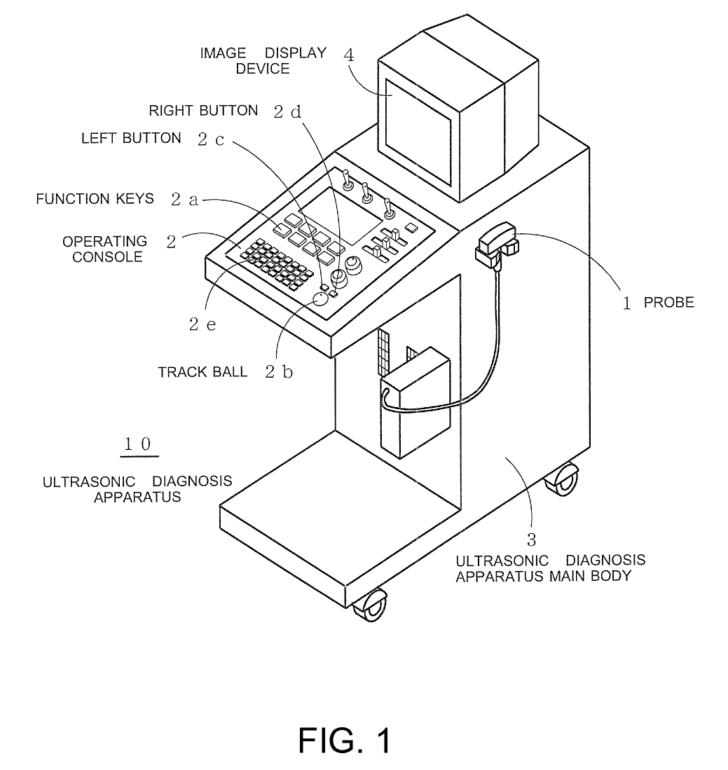

FIG. 1 is a perspective view of an ultrasonic diagnosis apparatus according to one embodiment.

This ultrasonic diagnosis apparatus 10 includes a probe 1 for transmitting and receiving ultrasonic waves, an operating console 2 allowing an operator to input a command and data, an ultrasonic diagnosis apparatus main body 3 that performs processing such as a trace measurement process which will be described later, and an image display device 4 for displaying an ultrasonic image.

The operating console 2 is equipped with function keys 2a including a freeze key, a trace measurement key, and a zoom-in parameter change key, a track ball 2b and its left button 2c and right button 2d attached thereto, a keyboard 2e, and others.

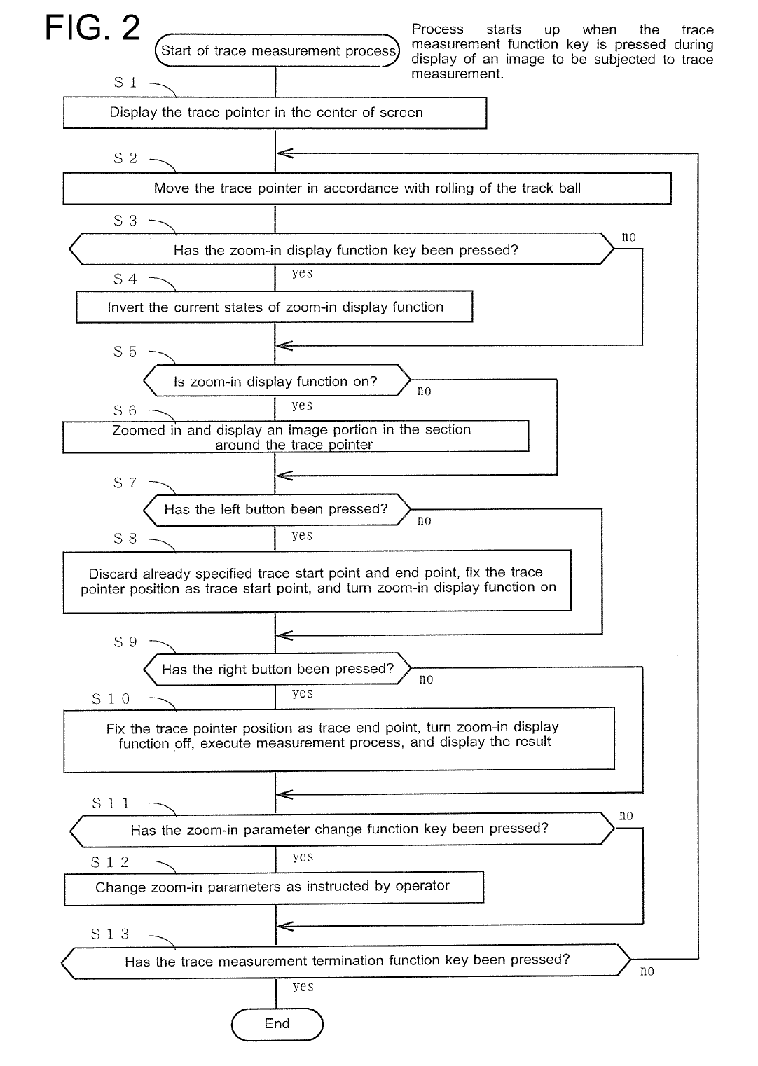

FIG. 2 is a flowchart illustrating a trace measurement process according to one embodiment.

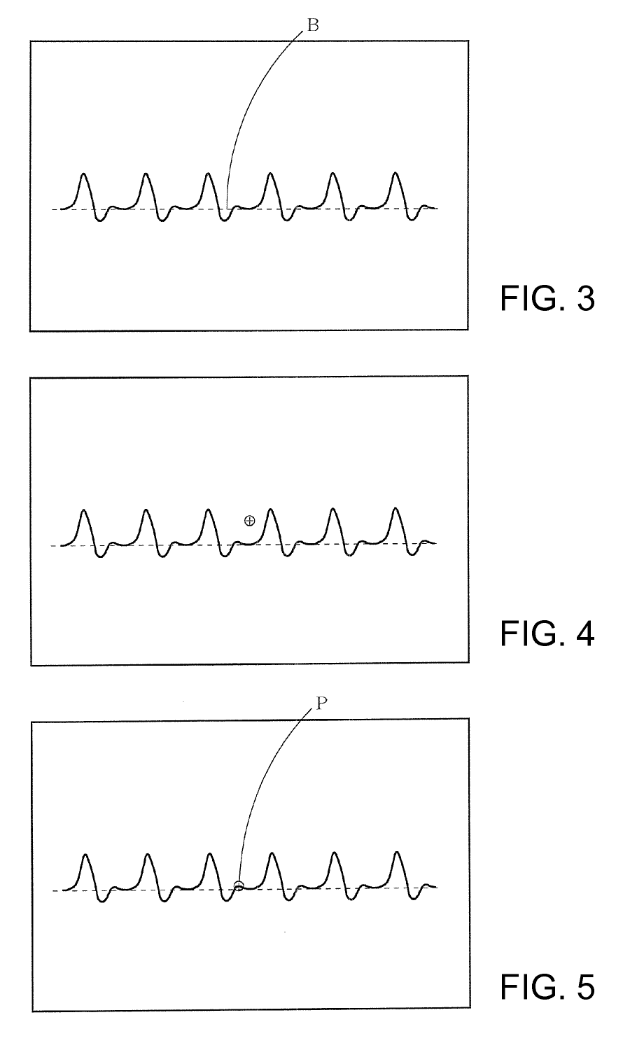

This trace measurement process displays an image that is subjected to trace measurement, as is depicted in FIG. 3, on the image display device 4. This process is frozen by the “freeze key” that is one of the function keys 2a, and activated by pressing the “trace measurement key” that is one of the function keys 2a when trace measurement is not performed.

The image shown in FIG. 3 is a Doppler spectrum, where B is a baseline.

At step S1, a trace pointer P is displayed at a default position in the screen, as is depicted in FIG. 4. The default position is, for example, a center position of the window in which the image is displayed.

At step S2, the trace pointer P is moved in accordance with rolling of the track ball 2b. The operator rolls the track ball 2b to move the trace pointer P to a trace start point, for example, as is depicted in FIG. 5.

At step S3, it is checked whether a “zoom-in display function key” which is one of the function keys 2a is pressed. If this key is pressed, the process goes to step S4; if not, the process goes to step S5.

At step S4, the zoom-in display function is switched to on if it is off at the present moment or switched to off if it is on at the present moment.

At step S5, the process goes to step S6 if the zoom-in display function is on at the present moment or goes to step S7 if off.

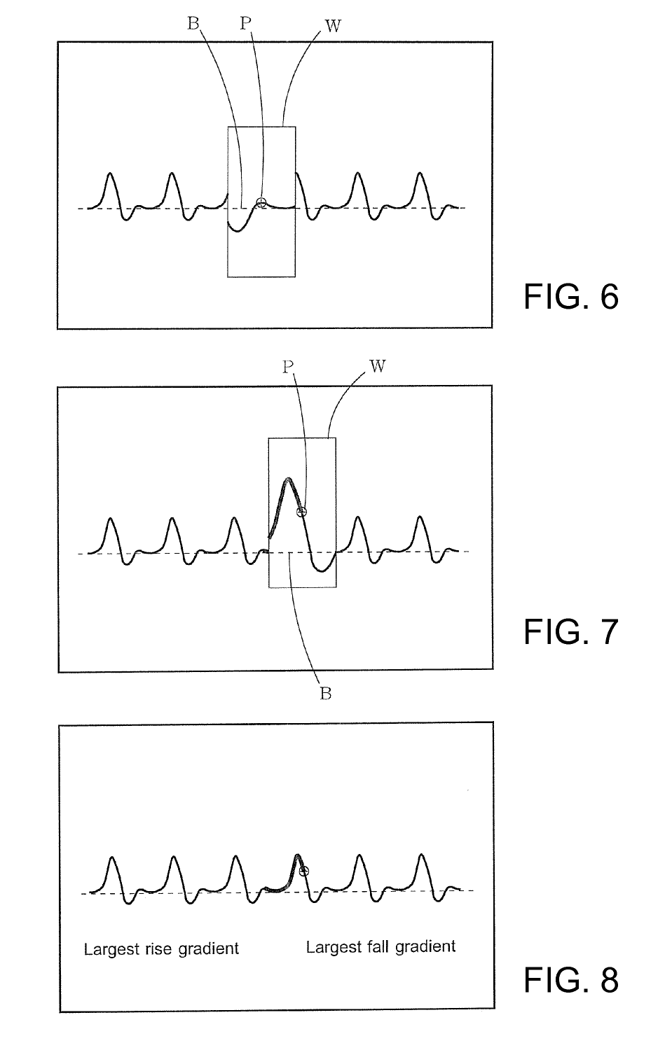

At step S6, a zoom-in display window W which is a rectangle with predetermined dimensions around the trace pointer is displayed, as is depicted in FIG. 6, wherein the scale factor at which the image is displayed within the zoom-in display window is set larger than the scale factor of display outside the window.

In the case of an image in which “time” is plotted on the abscissa like the Doppler spectrum, it is preferable to not to move the horizontal position of the trace pointer P before and after the display of the zoom-in display window W and move the vertical position of the trace pointer P so that the baseline B is consistent within and outside the zoom-in display window W. On the other hand, in the case of a B-mode image, it is preferable not to move both the horizontal and vertical positions of the trace pointer P before and after the display of the zoom-in display window W.

At step S7, it is checked whether the left button 2c attached to the track ball 2b has been pressed. If this button has been pressed, the process goes to step S8; if not, the process goes to step S9. The operator presses the left button 2c after moving the trace pointer P to the trace start point.

At step S8, the position of the trace pointer P is fixed as the trace start point. If a trace start point has already been specified, the previous trace start point is discarded. If a trace end point has already been specified, the trace end point is discarded. If the zoom-in display function is off at the present moment, it is changed to on. In addition, the track of trace pointer P after the trace start point has been defined is shown in a fashion in which it can be distinguished from the image, as is depicted in FIG. 7. For example, the track is shown as a line with a different thickness or color from the image.

At step S9, it is checked whether the right button 2d attached to the track ball 2b has been pressed. If this button has been pressed, the process goes to step S10; if not, the process goes to step S11. The operator presses the right button 2d after tracing the image contour with the trace pointer P and moving the trace pointer P to a trace end point.

At step S10, the position of the trace pointer P is fixed as the trace end point. If the zoom-in display function is on at the present moment, it is changed to off, and, as is depicted in FIG. 8, a trace measurement is executed based on the track of the trace pointer P from the trace start point to the trace end point and its result (a largest rise gradient and a largest fall gradient in FIG. 8) is displayed. After the trace end point has been defined, the track of the trace pointer P is not displayed.

At step S11, it is checked whether the “zoom-in parameter change key” which is one of the function keys 2a has been pressed. If this key has been pressed, the process goes to step S12; if not, the process goes to step S13.

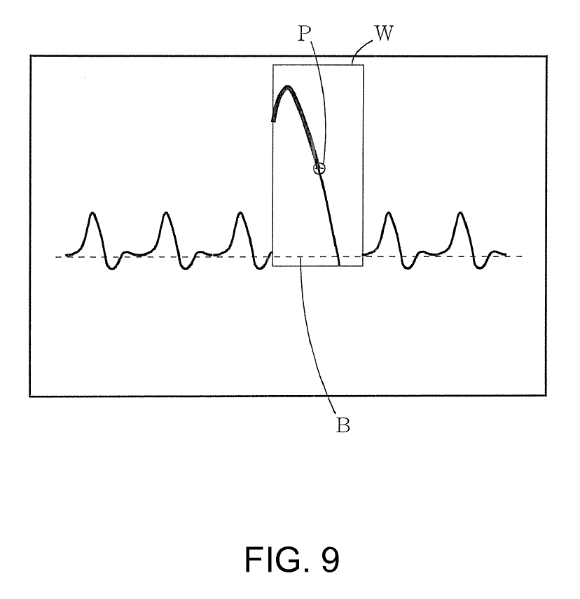

At step S12, zoom-in parameters are changed as instructed by the operator. For example, as is depicted in FIG. 9, the size of the zoom-in window W and the zoom-in factor can be changed.

At step S13, it is checked whether the “trace measurement key” which is one of the function keys 2a has been pressed. If this key has been pressed, the process terminates; if not, the process returns to step S2.

According to the ultrasonic diagnosis apparatus 10 of the embodiment, an image portion in a section around the trace pointer P is displayed at a higher scale factor than other parts of the image. Thus, it is easy to see whether the trace pointer P accurately traces the image contour or the like at the moment. Image portions before and after the section around the trace pointer P are displayed at a lower scale factor than the scale factor applied in the section around the trace pointer P. Thus, it is easy to find where the trace pointer P is now positioned in the entire image. Consequently, it is possible to improve the workability of manually tracing the contour of an image and the like displayed on screen.

The invention can be used for manual trace measurement of a Doppler spectrum and the like.

Many widely different embodiments of the invention may be configured without departing from the spirit and the scope of the present invention. It should be understood that the present invention is not limited to the specific embodiments described in the specification, except as defined in the appended claim.

1. An ultrasonic diagnosis apparatus comprising:
an image display device configured to display on a screen an image generated based on ultrasonic echo signals received from an subject;
a trace pointer display device configured to display a trace pointer on the screen during a trace measurement; and
an operating device configured to enable an operator to perform an action to instruct to start a trace measurement, an action to move the trance pointer on the screen, and an action to instruct to terminate the trace measurement, wherein said image display device is further configured to perform a zoomed-in display of an image portion in a section around the trace pointer.

2. The ultrasonic diagnosis apparatus according to claim 1, wherein the action to instruct to start a trace measurement is pressing a function key.

3. The ultrasonic diagnosis apparatus according to claim 1, wherein the action to move the trace pointer is rolling a track ball.

4. The ultrasonic diagnosis apparatus according to claim 1, wherein the action to terminate the trace measurement is pressing a function key.

5. The ultrasonic diagnosis apparatus according to claim 1, wherein an area of the zoomed-in display of an image portion moves within the screen according to movement of the trace pointer.

6. The ultrasonic diagnosis apparatus according to claim 1, further comprising a zoomed-in display area changing device configured to enable the operator to change dimensions of the area of the zoomed-in display of an image portion.

7. The ultrasonic diagnosis apparatus according to claim 5, further comprising a zoomed-in display area changing device configured to enable the operator to change dimensions of the area of the zoomed-in display of an image portion.

8. The ultrasonic diagnosis apparatus according to claim 1, wherein an area of the zoomed-in display of an image portion is a rectangular area that includes a position at which the trace pointer is displayed.

9. The ultrasonic diagnosis apparatus according to claim 1, wherein said operating device is configured to turn on/off a function for the zoomed-in display of an image portion.

10. The ultrasonic diagnosis apparatus according to claim 5, wherein said operating device is configured to turn on/off a function for the zoomed-in display of an image portion.

11. The ultrasonic diagnosis apparatus according to claim 9, wherein the action to turn on/off the function for the zoomed-in display of an image portion is pressing a function key.

12. The ultrasonic diagnosis apparatus according to claim 1, wherein said operating device is configured to specify a trace start point and to specify a trace end point.

13. The ultrasonic diagnosis apparatus according to claim 12, wherein the action to specify a trace start point is pressing a button attached to a track ball.

14. The ultrasonic diagnosis apparatus according to claim 12, wherein the action to specify a trace end point is pressing a button attached to a track ball.

15. The ultrasonic diagnosis apparatus according to claim 12, wherein said image display device is configured to start the zoomed-in display of an image portion, when the action to specify a trace start point is performed during an inactive state of the zoomed-in display of an image portion.

16. The ultrasonic diagnosis apparatus according to claim 12, wherein the said image display device is configured to deactivate the zoomed-in display of an image portion, when the action to specify a trace end point is performed during an active state of the zoomed-in display of an image portion.

17. The ultrasonic diagnosis apparatus according to claim 1, wherein said image display device is configured to start the zoomed-in display of an image portion, when the action to instruct to start a trace measurement is performed during an inactive state of the zoomed-in display of an image portion.

18. The ultrasonic diagnosis apparatus according to claim 1, wherein said image display device is configured to deactivate the zoomed-in display of an image portion, when the action to instruct to terminate a trace measurement is performed during an active state of the zoomed-in display of an image portion.

19. The ultrasonic diagnosis apparatus according to claim 1, wherein the image is a Doppler spectrum.

20. The ultrasonic diagnosis apparatus according to claim 1, wherein the image is a B-mode image.
